(19) United States
(12) Patent Application Publication
(10) Pub. No.: US 2014/0128688 A1
(43) Pub. Date: May 8, 2014

(54) Physiological Electrical Signal and Living Organism Movement Signal Sensing Apparatus

(71) Applicants: Min-Hsien Wu, Kaohsiung City (TW); Yi-Yuan Chiu, New Taipei City (TW); Hsin-Yao Wang, Chiayi City (TW); Song-Bin Huang, New Taipei City (TW)

(72) Inventors: Min-Hsien Wu, Kaohsiung City (TW); Yi-Yuan Chiu, New Taipei City (TW); Hsin-Yao Wang, Chiayi City (TW); Song-Bin Huang, New Taipei City (TW)

(21) Appl. No.: 14/055,926

(22) Filed: Oct. 17, 2013

(30) Foreign Application Priority Data
Oct. 19, 2012 (TW) 101138580

(51) Int. Cl. A61B 5/0205; A61B 5/11; A61B 5/04; A61B 5/0408; A61B 8/08; A61B 5/0492; A61B 5/0496; A61B 5/113; A61B 5/00; A61B 8/00; A61B 8/02; A61B 5/0478

(52) U.S. Cl. CPC A61B 5/0205; A61B 8/02; A61B 5/1126; A61B 5/04001; A61B 5/04087; A61B 5/0478; A61B 5/0492; A61B 5/0496; A61B 5/113; A61B 5/1107; A61B 5/0006; A61B 8/56; A61B 5/04012; A61B 8/54; A61B 8/52
USPC 600/301

(57) ABSTRACT

A physiological electrical signal and living organism movement signal sensing apparatus includes at least one electrode element, a piezoelectric sensing layer, a connecting layer and a control unit. The connecting layer is connected to the at least one electrode element, and the electrode element measures a physiological electrical signal of a living organism to generate a physiological sensing signal and the piezoelectric sensing layer measures a living organism movement signal to generate a living organism movement sensing signal, and the control unit receives the physiological sensing signal and the living organism movement sensing signal to determine and display the physiological status and movement of the living organism. The sensing apparatus has the features of providing highly integrated functions and simple structure.

### FIELD OF THE INVENTION

The present invention relates to a physiological electrical signal and living organism movement signal sensing apparatus, in particular to the sensing apparatus that uses an electrode element to measure a physiological electrical signal and a piezoelectric element to measure a living organism movement signal.

### BACKGROUND OF THE INVENTION

Heart muscle is the only human muscle with spontaneous beating and rhythmic contraction, and a cardiac conduction system sends out a electrical wave to excite fibers of the heart muscle to be stretched and contracted, and the electrical wave is produced and conducted to produce a feeble current, and thus electrodes of an electrocardiogram recorder is generally connected to a position proximate to a user's heart. Preferably, the magnitude of the surface electric potential difference between two points of a skin surface can be measured to plot an electrocardiogram.

The breathing movement of the living organism can be determined by using the piezoelectric sensing element, wherein the chest or abdominal muscle is stretched and contracted to cause a skin deformation when breathing, and the piezoelectric sensing element attached on the skin receives the skin deformation and converts the skin deformation into an electrical signal. In addition, the body movement can be measured by using the piezoelectric element and determined to be the movement of walking, waving hands, turning head, bowing, jumping, or squatting.

However, the prior art has the shortcoming of requiring the use of two different sensing apparatuses including an electrocardiogram electrode and a piezoelectric element to sense an electrocardiogram and a movement signal respectively. The prior art not only complicates the practical operation, but also causes inconvenience to users. In particular, each sensing apparatus is attached onto a body surface, and each sensing apparatus requires an independent connecting wire for transmitting an electrical signal to a data processing and displaying device, so that many apparatuses and different conductive wires used for measuring the signals are attached all over an examinee's body.

### SUMMARY OF THE INVENTION

Therefore, it is a primary objective of the present invention to overcome the aforementioned problem of the prior art by providing a physiological electrical signal and living organism movement signal sensing apparatus that can be attached onto a body surface of a living organism, and the sensing apparatus comprises at least one electrode element, a piezoelectric sensing layer, a connecting layer and a control unit. The electrode element measures a physiological electrical signal such as an electrocardiogram or an electroencephalogram to generate a physiological sensing signal. The piezoelectric sensing layer measures a living organism movement signal such as breathing or muscle contraction to generate a living organism movement sensing signal, and the piezoelectric sensing layer is coupled to the connecting layer, and the connecting layer is coupled to the at least one electrode element, but the electrode element is not contacted with the piezoelectric sensing layer, and the control unit is electrically coupled to the at least one electrode element and the piezoelectric sensing layer for receiving a physiological sensing signal from the electrode element and a living organism movement sensing signal from the piezoelectric sensing layer, and producing sensing result information after the signals are processed.

The control unit includes a memory for storing the sensing result information, and further transmits the sensing result information to an external control unit via a cable or wireless transmission, so that the external control unit can determine a physiological status and a movement of the living organism according to the sensing result information transmitted from the control unit, and can display the sensing result information in form of graphics, curves, symbols or texts.

The piezoelectric sensing layer is a sheet, wire or cable, and the sheet piezoelectric sensing layer is sandwiched between the cover layer and a part or the whole of the connecting layer and laminated tightly to enhance the intensity of the sensed piezoelectric signal, or the piezoelectric element is a sheet, wire, or cable having at least a warp, wherein the warp is provided for enhancing the intensity of the piezoelectric signal and increasing the overall tensile strength of the sensing apparatus, so as to improve the stability of sensing living organism movement signal.

Wherein, the sensing apparatus of the present invention further comprises a power supply unit for supplying electric power to the control unit.

Therefore, the sensing apparatus of the present invention has the advantages of high integration and simple structure, while providing both physiological electrical signal and living organism movement signal of the living organism and allowing users to observe a physiological status of the living organism conveniently. The present invention is especially applicable for recording data and performing instant remote physiological measurements by patients.

### BRIEF DESCRIPTION OF THE DRAWINGS

FIG. 1 is a schematic view of a physiological electrical signal and living organism movement signal sensing apparatus of a preferred embodiment of the present invention.

FIG. 2 is a schematic view of a physiological electrical signal and living organism movement signal sensing apparatus of another preferred embodiment of the present invention.

FIG. 3 is another preferred embodiment schematic view of a physiological electrical signal and living organism movement signal sensing apparatus of the present invention.

### DESCRIPTION OF THE PREFERRED EMBODIMENTS

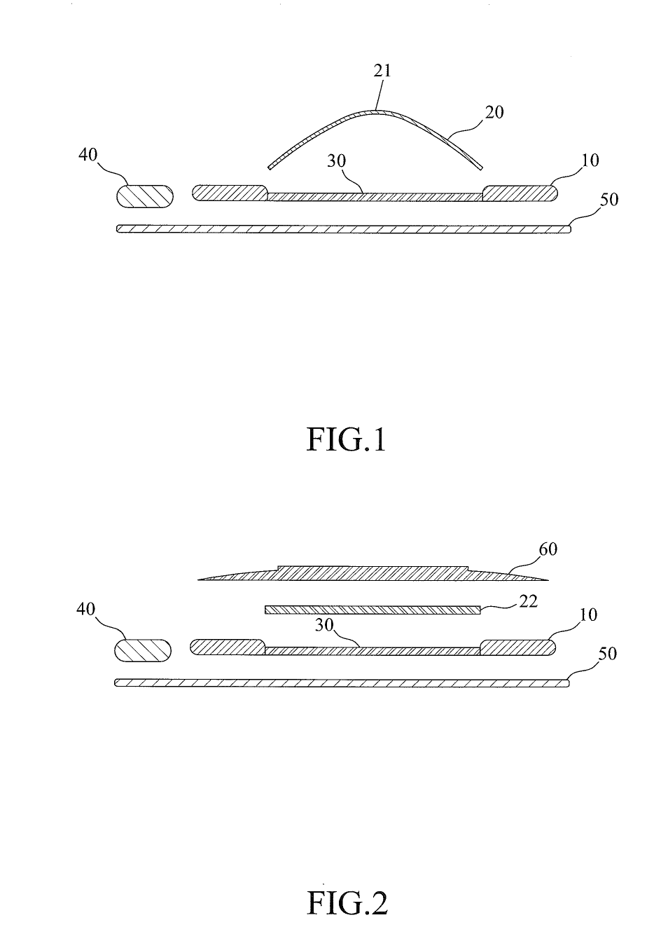

With reference to FIG. 1 for a physiological electrical signal and living organism movement signal sensing apparatus of the present invention, the sensing apparatus comprises at least one electrode element 10, a piezoelectric sensing layer 20, a connecting layer 30 and a control unit 40, and the sensing apparatus of the present invention can be attached onto a body surface 50 of a living organism such as human chest surface, abdominal surface, limb surface or head surface, or animal skin.

Wherein, the number of electrode elements 10 can be any positive integer.

The electrode element 10 has the functions of sensing and transmitting a voltage or current signal on the body surface 50 of the living organism, so that the electrode element 10 can be used for measuring a physiological electrical signal such as an electrocardiogram, an electroencephalogram, an electromyogram, an electroneurogram, an electroretinogram, an electrogastrography, an electroneuromyography, an electrocorticogram, an electrooculography and an electronysagmography of the living organism to generate a corresponding physiological sensing signal. The electrode element 10 is made of an electrically conductive material such as metal, alloy, conductive plastic or conductive ceramic.

The piezoelectric sensing layer 20 has the function of sensing a deformation of the body surface 50 to generate an electrical signal, and the piezoelectric sensing layer 20 is made of a piezoelectric material such as electromechanical film, polyamide, PbTiO₃, quartz, SiO₂, LiNbO₃, LiTaO₃, BaTiO₃, Pb(Zr,Ti)O₃, GaAs, AlN, ZnO, BiFeO₃, polyvinylidene fluoride, polyethylene terephthalate, polyethylene, polypropylene, polyvinyl chloride, polystyrene, polytetrafluoroethene, polymethylmethacrylate or polydimethylsiloxane. The piezoelectric sensing layer 20 is in the shape of a sheets having at least a warp 21 as shown in the middle part of the piezoelectric sensing layer 20 of FIG. 1, wherein the warp 21 is provided for increasing the deformation of the piezoelectric sensing layer 20 to further enhance the intensity of the generated piezoelectric signal and the overall elongation of the sensing apparatus, so as to improve the stability of sensing the living organism movement signal.

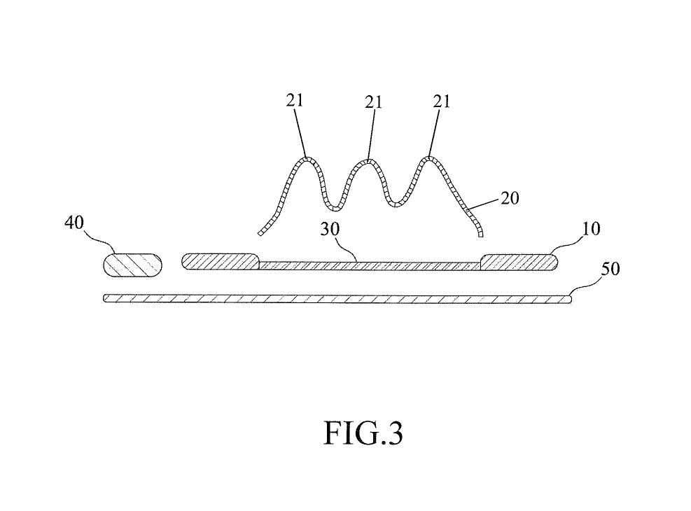

With reference to FIG. 3 for a physiological electrical signal and living organism movement signal sensing apparatus in accordance with another preferred embodiment of the present invention, the sensing apparatus comprises at least one electrode element 10, a piezoelectric sensing layer 20, a connecting layer 30 and a control unit 40, and the sensing apparatus of the present invention can be attached onto a body surface 50 of a living organism such as human chest surface, abdominal surface, limb surface or head surface, or animal skin. The piezoelectric sensing layer 20 is in the shape of a sheets having three warps 21 as shown in the middle part of the piezoelectric sensing layer 20 of FIG. 3, wherein the warp 21 is provided for increasing the deformation of the piezoelectric sensing layer 20 to further enhance the intensity of the generated piezoelectric signal and the overall elongation of the sensing apparatus, so as to improve the stability of sensing the living organism movement signal.

Therefore, the piezoelectric sensing layer 20 can be used for measuring a living organism movement signal generated by the deformation of the body surface 50 that is caused by contracting the muscles of the living organism in an activity such as breathing, heartbeat, body movement, skin stretching and contraction, muscle contraction, talking, bowing, head turning, hand swinging and walking, and generating a corresponding living organism movement sensing signal.

The connecting layer 30 is electrically insulating and capable of connecting the at least one electrode element 10 to provide the electrical insulation and fixed connection effects, and particularly capable of enhancing the overall rigidity of the sensing apparatus of the present invention. The connecting layer 30 is made of an electrically insulating material such as polyamide, acrylic, acrylonitrile-butadiene-styrene, phenolic resin, epoxy, polyester, silicone, polyurethane (PU), latex, rubber, glass, food grade silicone, polyethylene terephthalate, polyethylene, polyproylene, polyvinyl chloride, polystyrene, polyvinylidene fluoride, polytetrafluoroethene, polymethylmethacrylate or polydimethylsiloxane.

The control unit 40 is electrically coupled to the at least one electrode element 10 and the piezoelectric sensing layer 20 for receiving a physiological sensing signal from the electrode element 10 and a living organism movement sensing signal from the piezoelectric sensing layer 20 and generating sensing result information simultaneously after the signals are processed. Further, the sensing result information is transmitted to an external control unit via a cable or wireless transmission, so that the external control unit can determine the physiological status and movement of the living organism such as heartbeat or breathing, and display them in form of graphics, curves, symbols or texts according to the sensing result information transmitted from the control unit 40.

The signal processing of the control unit 40 includes the functions of filtering, amplification, analog-to-digital conversion and/or digital-to-analog conversion for filtering noises, amplifying useful signals, converting analog and digital signals, and driving the electrode element 10 or the piezoelectric sensing layer 20. Further, the control unit 40 includes a memory for storing the generated sensing result information.

In addition, the control unit 40 has the function of receiving an instruction transmitted from an external control unit, and executing a corresponding function according to the instruction. For instance, the instruction includes a read instruction and/or a drive instruction, wherein the control unit 40 transmits the generated sensing result information to an external control unit according to a corresponding function of the read instruction, and the sensing result information is read by the external control unit, and the control unit 40 drives the at least one electrode element 10 to generate a corresponding electrical signal according to a corresponding function of the drive instruction to perform an impedance pneumography measurement or a functional electrical stimulation, and/or drive the piezoelectric sensing layer 20 to produce a corresponding deformation.

Therefore, the external control unit can read the sensing result information stored in the control unit 40 via a remote control method to achieve the remote reading function. In addition, the external control unit can drive the electrode element 10 to generate an electrical signal, and further use the electrical signal to achieve the impedance pneumography measurement or the functional electrical stimulation.

The external control unit further drives the piezoelectric sensing layer 20 to produce a deformation, so as to provide a massaging function such as soothing excessively tight muscles.

In a preferred embodiment of the present invention, the sensing apparatus further comprises a power supply unit for supplying electric power to a controller, and the power supply unit includes a primary battery, a secondary battery, or a wireless power supply module.

With reference to FIG. 2 for a physiological electrical signal and living organism movement signal sensing apparatus in accordance with another preferred embodiment of the present invention, the sensing apparatus comprises at least one electrode element 10, a piezoelectric sensing layer 22, a connecting layer 30, a control unit 40 and a cover layer 60, and the sensing apparatus can be attached onto the body surface 50 of the living organism.

In FIG. 2, the sensing apparatus further comprises a cover layer 60, and the piezoelectric sensing layer 22 is a flat sheet, a wire or a cable, and the piezoelectric sensing layer is not in a curved shape. The technical characteristics of the remaining same elements are the same, and thus will not be described again.

The cover layer is made of an electrically insulating material such as polyamide, acrylic, acrylonitrile-butadiene-styrene, phenolic resin, epoxy, polyester, silicone, polyurethane (PU), latex, rubber, glass, food grade silicone, polyethylene terephthalate, polyethylene, polyproylene, polyvinyl chloride, polystyrene, polyvinylidene fluoride, polytetrafluoroethene, polymethylmethacrylate or polydimethylsiloxane.

The cover layer 60 is disposed on the piezoelectric sensing layer 22, and the piezoelectric sensing layer 22 is sandwiched between the cover layer 60 and a part or the whole of the connecting layer 30 to laminate the piezoelectric sensing layer 22 tightly, so as to enhance the intensity of the sensed piezoelectric signal.

To enhance the electromagnetic shielding effect of the piezoelectric sensing layer 22 and prevent the electromagnetic interference caused by external electromagnetic waves, the invention further comprises two shielding layers for sandwiching the piezoelectric sensing layer 22 by a part or the whole of the shielding layers to provide the shielding effect, wherein the shielding layers are made of an electrically conductive metal material.

It is noteworthy that the single piezoelectric sensing layer, single connecting layer, single control unit, single cover layer, single external control unit and single power supply unit of the foregoing preferred embodiment are used for the purpose of illustrating the technical characteristics of the present invention only, but not intended for limiting the scope of the present invention. In other words, the number of piezoelectric sensing layers, connecting layers, control units, cover layers, external control units and power supply units of the present invention can be any positive integer. Similarly, the single physiological electrical signal, single living organism movement signal and single living organism movement sensing signal also cover the scope of a plurality of physiological electrical signals, a plurality of living organism movement signals and a plurality of living organism movement sensing signals.

The physiological electrical signal and living organism movement signal sensing apparatus of the present invention provides highly integrated functions and has the advantages of simple structure and miniaturization, while providing the function of sensing both physiological electrical signal and living organism movement signal. In addition, the sensing apparatus can be set on clothes, pants, diapers and lingerie conveniently to improve the convenience of use and allows medical professionals to conduct an impedance pneumography or a functional electrical stimulation for patients by a remote control method.

What is claimed is:

1. A physiological electrical signal and living organism movement signal sensing apparatus, attached on a body surface of a living organism, comprising:
at least one electrode element, attached onto the living organism for measuring at least one physiological electrical signal of the living organism to generate at least one physiological sensing signal;
at least one piezoelectric sensing layer, for measuring at least one living organism movement signal of the living organism to generate at least one living organism movement sensing signal;
at least one connecting layer, electrically insulating, and coupled to the at least one electrode element and the piezoelectric sensing layer;
at least one control unit, electrically coupled to the at least one electrode element and the at least one piezoelectric sensing layer, for receiving a physiological sensing signal from the electrode element and a living organism movement sensing signal from the piezoelectric element, and generating sensing result information after the signals are processed; and
at least one power supply unit, for supplying electric power to the at least one control unit.

2. The sensing apparatus of claim 1, wherein the sensing result information is stored in the control unit or transmitted to at least one external control unit.

3. The sensing apparatus of claim 1, wherein the piezoelectric sensing layer is a sheet, a wire or a cable having at least a warp which is made of a material selected from the group consisting of electromechanical film, polyamide, PbTiO₃, quartz, SiO₂, LiNbO₃, LiTaO₃, BaTiO₃, Pb(Zr,Ti)O₃, GaAs, AlN, ZnO, BiFeO₃, polyvinylidene fluoride, polyethylene terephthalate, polyethylene, polypropylene, polyvinyl chloride, polystyrene, polytetrafluoroethene, polymethyl methacrylate, and polydimethyl siloxane.

4. The sensing apparatus of claim 1, further comprising at least one cover layer made of an electrically insulating material selected from the group consisting of polyamide, acrylic, acrylonitrile-butadiene-styrene, phenolic resin, epoxy, polyester, silicone, polyurethane (PU), latex, rubber, glass, food grade silicone, polyethylene terephthalate, polyethylene, polyproylene, polyvinyl chloride, polystyrene, polyvinylidene fluoride, polytetrafluoroethene, polymethylmethacrylate and polydimethylsiloxane, and the piezoelectric sensing layer being a flat sheet, wire or cable, and the cover layer being disposed on the piezoelectric sensing layer, and the piezoelectric sensing layer being sandwiched between the cover layer and a part or the whole of the connecting layer, and the piezoelectric sensing layer being made of a material selected from the group consisting of electromechanical film, polyamide, PbTiO₃, quartz, SiO₂, LiNbO₃, LiTaO₃, BaTiO₃, Pb(Zr,Ti)O3, GaAs, AlN, ZnO, BiFeO₃, polyvinylidene fluoride, polyethylene terephthalate, polyethylene, polypropylene, polyvinyl chloride, polystyrene, polytetrafluoroethene, polymethyl methacrylate, polydimethyl siloxane.

5. The sensing apparatus of claim 1, wherein the physiological electrical signal includes at least one selected from the group consisting of an electrocardiogram, an electroencephalogram, an electromyogram, an electroneurogram, an electroretinogram, an electrogastrography, an electroneuromyography, an electrocorticogram, an electrooculography and an electronysagmography, and the living organism movement signal includes at least one selected from the group consisting of breathing, heartbeat, body movement, skin stretching and contraction, and muscle contraction.

6. The sensing apparatus of claim 2, wherein the control unit receives an instruction transmitted from the external control unit and executes a corresponding function according to the instruction, and the instruction comprises a read instruction or a drive instruction, and the control unit transmits the sensing result information to the external control unit according to the read instruction, and drives the at least one electrode element to generate an electrical signal, or drives the piezoelectric sensing layer to produce a corresponding deformation according to the drive instruction.

7. The sensing apparatus of claim 1, wherein the control unit includes a memory for storing the sensing result information, and transmits the sensing result information via a cable or wireless transmission.

8. The sensing apparatus of claim 7, wherein the control unit receives an instruction transmitted from an external control unit and executes a corresponding function according to the instruction, and the instruction comprises a read instruction or a drive instruction, and the control unit transmits the sensing result information stored in the memory from the control unit to the external control unit according to the corresponding function of the read instruction, and the control unit drives the at least one electrode element to generate a corresponding electrical signal, or drives the piezoelectric sensing layer to produce a corresponding deformation according to the corresponding function of the drive instruction.

9. The sensing apparatus of claim 8, wherein the memory stores driving information, and the control unit reads the driving information from the memory according to a corresponding function of the drive instruction, and drives the at least one electrode element to generate a corresponding electrical signal, or drives the piezoelectric sensing layer to produce a corresponding deformation according to the driving information.

10. The sensing apparatus of claim 1, further comprising at least one shielding layer with a part or the whole for sandwiching the piezoelectric sensing layer to provide a shielding effect, and the shielding layer is made of an electrically conductive metal material.

11. The sensing apparatus of claim 1, wherein the connecting layer made of an electrically insulating material selected from the group consisting of polyamide, acrylic, acrylonitrile-butadiene-styrene, phenolic resin, epoxy, polyester, silicone, polyurethane (PU), latex, rubber, glass, food grade silicone, polyethylene terephthalate, polyethylene, polyproylene, polyvinyl chloride, polystyrene, polyvinylidene fluoride, polytetrafluoroethene, polymethylmethacrylate and polydimethylsiloxane.
